(19) United States
(12) Patent Application Publication
(10) Pub. No.: US 2017/0014463 A1
(43) Pub. Date: Jan. 19, 2017

(54) Composition Comprising Plants Extracts that Synergistically Treat or Inhibit Pathological Conditions and Method of Making

(71) Applicant: Bruce Nolan, Ontario, CA (US)

(72) Inventor: Bruce Nolan, Ontario, CA (US)

(21) Appl. No.: 15/209,920

(22) Filed: Jul. 14, 2016

Related U.S. Application Data
(60) Provisional application No. 62/192,866, filed on Jul. 15, 2015.

(51) Int. Cl. A61K 36/68; A61K 36/534

(52) U.S. Cl. CPC A61K 36/68; A61K 36/534; A61K 2236/13; A61K 2236/15; A61K 2236/51; A61K 2236/331; A61K 2236/53

(57) ABSTRACT

A composition including plant extracts that synergistically treat or inhibit pathological conditions and a method for developing the composition is provided. The composition includes bioactive chemical agents extracted from the Plantago major plant species including, but not limited to, acetonide, catalpol, hispidulin, loliolide, neptin, rhamnose, and scutellarin, and bioactive chemical agents extracted from the Mentha spicata plant species including, but not limited to, cadinol, diosmetin, eugenol, furfural, hesperidin, luteolin, and piperitone. The method includes the steps of washing Plantago major and Mentha spicata plant material, freezing the plant material, cutting the frozen plant material into pieces, heating the plant material in purified water to form a resultant solution containing Plantago major and Mentha spicata plant extracts, cooling the resultant solution, and filtering the Plantago major and Mentha spicata plant material out of the resultant solution to produce the composition.

### CROSS REFERENCE TO RELATED APPLICATIONS

This application claims the benefit of U.S. Provisional Application No. 62/192,866 filed on Jul. 15, 2015. The above identified patent application is herein incorporated by reference in its entirety to provide continuity of disclosure.

### BACKGROUND OF THE INVENTION

The present invention relates generally to synergistic compositions that treat or inhibit pathological conditions in the body, such as cancer, Alzheimer's disease, benign prostate hyperplasia, and cardiovascular disease, and to methods of producing the compositions. More specifically, the invention relates to a composition comprising bioactive agents extracted from plants of the species Plantago major and Mentha spicata.

Pathological conditions are those caused by diseases, such as cancer. Cancer is a general term frequently used to indicate any of the various types of malignant neoplasms (i.e. abnormal tissue that grows by cellular proliferation more rapidly than normal), most of which invade surrounding tissue, may metastasize to several sites, are likely to recur after attempted removal, and cause death unless adequately treated (Stedman's Medical Dictionary, Williams & Wilkins, Baltimore, Md., 26th ed. 1995). Although a variety of approaches to cancer therapy, including surgical resection, radiotherapy, and chemotherapy, have been available and commonly used for many years, cancer remains one of the leading causes of death in the world.

A large number of chemotherapeutic agents have been developed, however, many of these are associated with undesirable side-effects. In addition, in some cases, specific patient subgroups, such as elderly patients and patients suffering from obesity or neutropenia, exhibit an intolerance for standard or optimal chemotherapeutic doses and as a result receive sub-optimal doses of chemotherapeutics during cancer treatments (Griggs J J, Sorbero M E S, Lyman G H (2005), Arch. Inter. Med., 165(11):1267-73; Colleoni M, Gelber R D et al. (2005), Lancet, 366(9491):1108-10, Madarnas Y, et al. (2001), Breast Cancer Res Treat, 66(2):123-33, and Lyman G H, Dale D C, Crawford J. (2003), J. Clin. Oncol., 21(24):4524-31). As demonstrated by Griggs et al., administration of these sub-optimal doses of chemotherapeutics to obese women afflicted with breast cancer resulted in a poor outcome. In this case optimal doses, which were based on the patient's body size, could not be administered to overweight individuals in light of the toxic effects associated with the high doses on organs. Currently, higher chemotherapeutic dosing may be facilitated by administration of the adjuvant Neupogen®. Here, faster recovery of white blood cells may permit a patient to withstand a higher dose of chemotherapy.

Moreover, a large number of preventative dietary supplements have been developed for the purpose of inhibiting or preventing cancer and other pathological diseases. However, none of these dietary supplements have plants extracts that provide synergistic immunomodulatory effects on the immune system that inhibit the onset of pathological diseases in the body.

The use of plant extracts or components of plant extracts for the treatment of cancer or for inhibiting angiogenesis has been described. For example, a synergistic composition of lignans obtained from the plant extract of Cedrus deodra that exhibit anticancer activities for breast, cervix, neuroblastoma, colon, liver, lung, mouth, ovary and prostate cancer is known in the art. Further, plant extracts comprising oleouropein to inhibit angiogenesis are known in the art. Additionally, herbal plant extracts of the Anoectochilus family of plants and in particular the Anoectochilus formosanus species, and their use for chemo-prevention, or complementary/alternative control of various human malignant diseases are known in the art. Moreover, plant extracts comprising a chemical agent of the diterpene family obtained from a member of the Euphorbiaceae family of plants for use in the treatment or prophylaxis of prostate cancer or a related cancer or condition are known in the art. Lastly, plant extracts from Euphorbaciae obesa and their use for inducing apoptosis and growth inhibition of a cancerous cell are known in the art.

While these compositions fulfill their respective, particular objectives and requirements, the aforementioned patents and patent publications do not disclose a composition having plant extracts comprising bioactive chemical agents obtained from a member of the Plantaginaceae family of plants and a member of the Lamiaceae family of plants to synergistically treat and inhibit pathological conditions.

In these respects, the composition according to the present invention substantially departs from the conventional compositions and methods of the prior art, and in so doing provides a composition and method for developing the composition primarily developed for the purpose of providing a composition comprising plant extracts that synergistically treat or inhibit pathological conditions.

### SUMMARY OF THE INVENTION

In view of the foregoing disadvantages inherent in the known types of compositions comprising plant extracts now present in the prior art, the present invention provides a composition comprising plant extracts from the Plantaginaceae plant family and the Lamiaceae plant family wherein the same can be utilized for treating or inhibiting the development of pathological conditions. The present system comprises a composition for inhibiting or treating pathological conditions, the composition comprising an effective amount of a plurality of plant extracts including acetonide, adenine, allantoin, apigenin, apigenin-7-glucoside, asperuloside, aucubin, baicalein, baicalin, benzoic acid, caffeic acid, catalpol, chlorogenic acid, cinnamic acid, D-glucose, D-xylose, ferulic acid, fumaric acid, geniposidic acid, gentisic acid, glucoraphanin, hispidulin, hydroxycinnamic acid, loliolide, luteolin, luteolin-7-O-beta-D-glucoside, neochlorogenic acid, nepetin, oleanolic acid, P-coumaric acid, P-hydroxybenzoic acid, phenolcarbonic acid, rhamnose, saccharose, salicylic acid, scutellarin, sitosterol, succinic acid, sulforaphane, syringic acid, syringin, tyrosol, ursolic acid, vanillic acid, acetic acid, acetoin, acetone, alpha-humulene, alpha-methyl-P-methylstyrene, alpha-pinene, alpha-terpineol, alpha-thujene, arginine, benzaldehyde, benzyl alcohol, benzyl isobutyrate, beta-bourbonene, beta-caryophyllene, beta-caryophyllene oxide, beta-elemene, beta-ionone, beta-limonene-1,2-oxide, beta-phenethyl alcohol, beta-phenethyl methyl ether, beta-pinene, beta-sitosterol, borneol, butyric acid, cadinol, camphene, carvacrol, L-carvone, carvone-10-acetate, carvone-8,9-oxide, chloroform, chlorophyll-A, chlorophyll-B, cineole, cis-carveol, cis-carvone oxide, cis-carvyl acetate, cis-jasmone, decan-1-ol, dehydrocarvacrol, delta-cadinene, diacetyl, dimethyl sulfide, dimethyl sulfoxide, diosmetin, diosmetin-7-glucoside, diosmin, ethanol, ethyl-N-valerate, eugenol, furfural, geraniol, heptan-3-one, hesperidin, hexan-1-ol, isoamyl isovalerate, isoleucine, jasmone, leucine, luteolin-7-O-beta-D-glucuronide, lysine, menthofuran, menthol, menthol acetate, methanol, methionine, myrtenal, myrtenol, neomenthol, niacin, oxalic acid, P-cymol, perillaldehyde, perillyl alcohol, phenethyl alcohol, piperitone, piperitone oxide, P-menth-trans-2,8-dien-1-ol, pomolic acid, propionaldehyde, riboflavin, rosmarinic acid, thiamin, threonine, thymol, tiglaldehyde, toluene, trans-carveol, trans-jasmone, terpinen-4-ol, valeric acid, valine, and vanillin.

Further the present invention comprises a method of producing a composition for inhibiting or treating pathological conditions, comprising an effective amount of a plurality of plant extracts from a Plantago major plant species and an effective amount of plurality of plant extracts from a Mentha spicata plant species, the method comprising the steps of: washing Plantago major plant material and Mentha spicata plant material with purified water; removing excess water from the Plantago major plant material and the Mentha spicata plant material; freezing the Plantago major plant material and the Mentha spicata plant material; cutting the Plantago major plant material and the Mentha spicata plant material into fragments; heating the Plantago major plant material and the Mentha spicata plant material in purified water to form a resultant solution including Plantago major and Mentha spicata plant extracts; cooling the resultant solution; pressing the Plantago major plant material and the Mentha spicata plant material remaining in the resultant solution; and filtering the fragmented plant material from the resultant solution.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a list of the bioactive chemical agents extracted from the Plantago major and Mentha spicata plant species which are present in the composition.

FIG. 2 shows a flow chart of the method of producing the composition.

### DETAILED DESCRIPTION OF THE INVENTION

Reference is made herein to the attached drawings. Like reference numerals are used throughout the drawings to depict like or similar elements of the composition and method. The figures are intended for representative purposes only and should not be considered to be limiting in any respect.

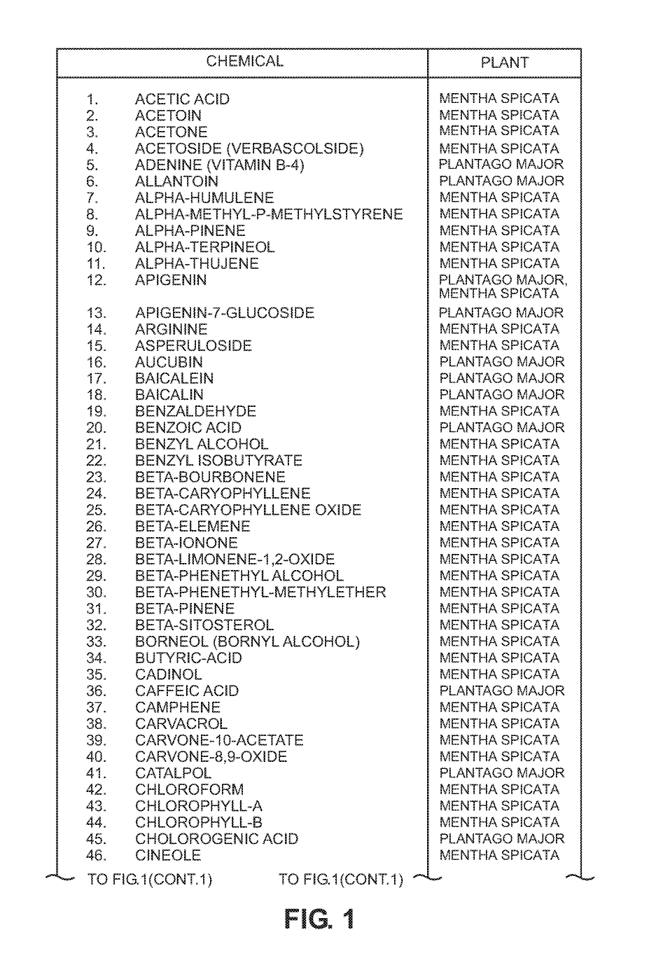

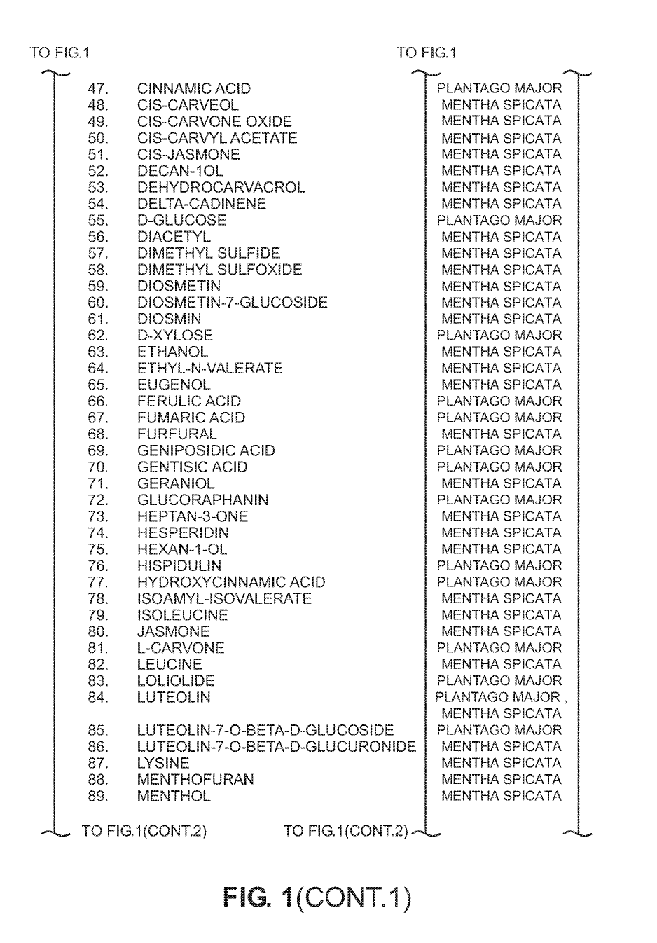

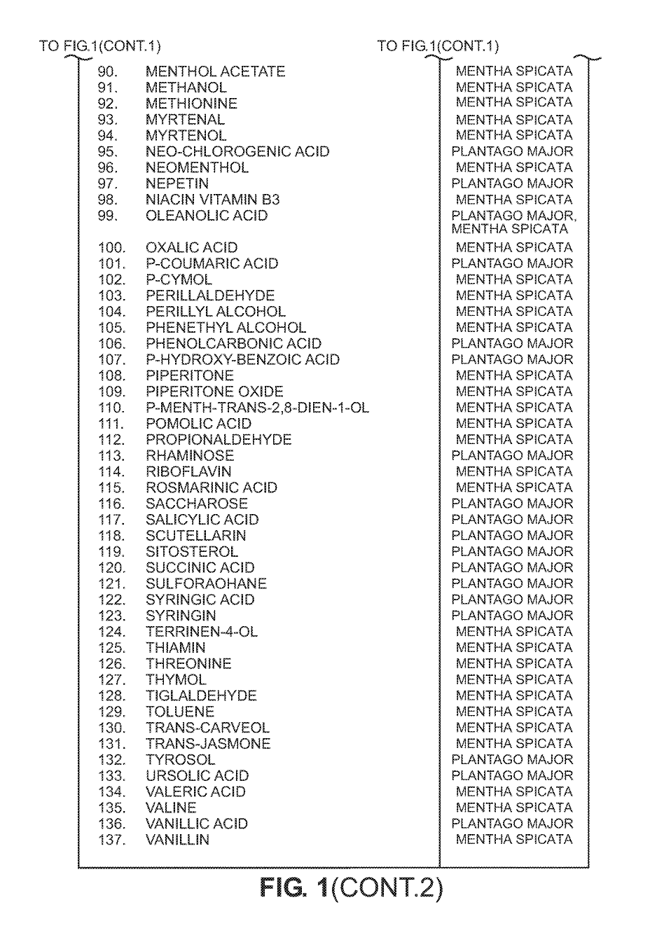

Referring now to FIG. 1, there is shown a list of the bioactive chemical agents extracted from the Plantago major and Mentha spicata plant species which are present in the composition. The composition in accordance with the present invention comprises 44 bioactive chemical agents from the Plantago major species of the Plantaginaceae plant family and 96 bioactive chemical agents from the Mentha spicata species of the Lamiaceae plant family, three of which are present in both the Plantago major and Menth spicata species. Specifically, the composition comprises 137 different chemical agents, 12 of which are solvents. The chemical agents include acetonide, adenine, allantoin, apigenin, apigenin-7-glucoside, asperuloside, aucubin, baicalein, baicalin, benzoic acid, caffeic acid, catalpol, chlorogenic acid, cinnamic acid, D-glucose, D-xylose, ferulic acid, fumaric acid, geniposidic acid, gentisic acid, glucoraphanin, hispidulin, hydroxycinnamic acid, loliolide, luteolin, luteolin-7-O-beta-D-glucoside, neochlorogenic acid, nepetin, oleanolic acid, P-coumaric acid, P-hydroxybenzoic acid, phenolcarbonic acid, rhamnose, saccharose, salicylic acid, scutellarin, sitosterol, succinic acid, sulforaphane, syringic acid, syringin, tyrosol, ursolic acid, and vanillic acid, all of which are extracted from the Plantago major plant species, and acetic acid, acetoin, acetone, alpha-humulene, alpha-methyl-P-methylstyrene, alpha-pinene, alpha-terpineol, alpha-thujene, apigenin, arginine, benzaldehyde, benzyl alcohol, benzyl isobutyrate, beta-bourbonene, beta-caryophyllene, beta-caryophyllene oxide, beta-elemene, beta-ionone, beta-limonene-1,2-oxide, beta-phenethyl alcohol, beta-phenethyl methyl ether, beta-pinene, beta-sitosterol, borneol, butyric acid, cadinol, camphene, carvacrol, L-carvone, carvone-10-acetate, carvone-8,9-oxide, chloroform, chlorophyll-A, chlorophyll-B, cineole, cis-carveol, cis-carvone oxide, cis-carvyl acetate, cis-jasmone, decan-1-ol, dehydrocarvacrol, delta-cadinene, diacetyl, dimethyl sulfide, dimethyl sulfoxide, diosmetin, diosmetin-7-glucoside, diosmin, ethanol, ethyl-N-valerate, eugenol, furfural, geraniol, heptan-3-one, hesperidin, hexan-1-ol, isoamyl isovalerate, isoleucine, jasmone, leucine, luteolin, luteolin-7-O-beta-D-glucuronide, lysine, menthofuran, menthol, menthol acetate, methanol, methionine, myrtenal, myrtenol, neomenthol, niacin, oleanolic acid, oxalic acid, P-cymol, perillaldehyde, perillyl alcohol, phenethyl alcohol, piperitone, piperitone oxide, P-menth-trans-2,8-dien-1-ol, pomolic acid, propionaldehyde, riboflavin, rosmarinic acid, thiamin, threonine, thymol, tiglaldehyde, toluene, trans-carveol, trans-jasmone, terpinen-4-ol, valeric acid, valine, and vanillin all of which are extracted from the Mentha spicata plant species. In one embodiment, the composition further comprises purified water, such as distilled water, double distilled water, deionized water, or demineralized water, thereby forming an aqueous based solution having all of the aforementioned chemical agents.

The concentrations of the bioactive chemicals vary depending on the quantity of Plantago major and Mentha spicata quantities used during extraction. In one embodiment, the sum of all bioactive chemicals extracted from Plantago major plant material that are present in the composition is 0.239 grams. In another embodiment, the sum of all bioactive chemicals extracted from Mentha spicata plant material that are present in the composition is 0.019 grams. In yet another embodiment, the weight of all bioactive chemicals present in the composition comprises a total sum of 0.258 grams, 0.239 grams of bioactive chemicals extracted from Plantago major plant material and 0.019 grams of bioactive chemicals extracted from Mentha spicata plant material.

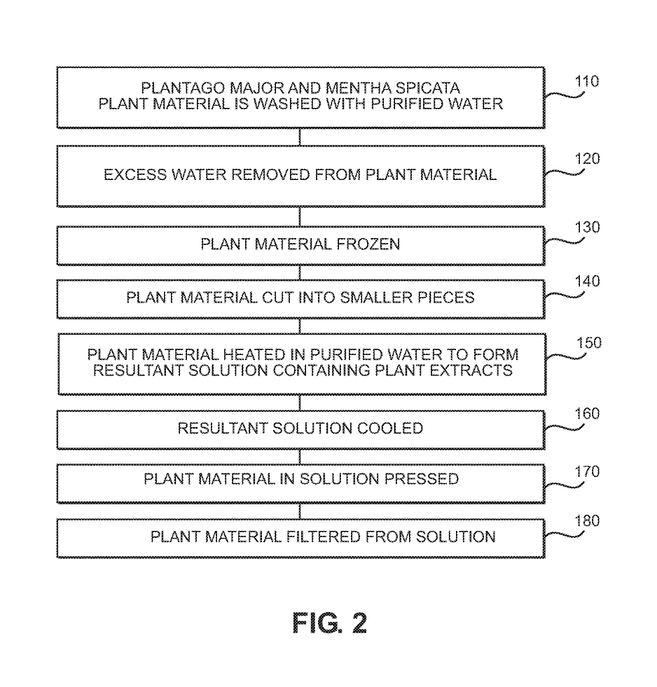

Referring now to FIG. 2, there is shown a flow chart of the method of producing the composition. The method of producing the above composition comprises extracting the aforementioned chemicals from Plantago major plants and Mentha spicata plants. During the entire extraction process, non-degradable gloves, such as nitrile gloves, are to be worn, such that there is no transfer of body oils and other possible contaminants from a user to the plant material while being prepared for extraction. In this method, only leaves of the Plantago major plant species and the Mentha spicata plant species are to be used. In one embodiment, approximately 25 grams of Plantago major and 2 grams of Mentha spicata are used. In another embodiment, the leaves are harvested and gathered for this method and only the fresh, green leaves are used. No roots, stems, flower stalks, or seeds are to be used in this method. In yet another embodiment, all of the plant material to be used is inspected for damage, disease, infestation, foreign debris, and residue before use and all damaged plant material is removed and not utilized for this method. The plant material to be used for this method is to be kept cool to prevent the wilting or degradation thereof. In one embodiment, the plant material to be used may be stored in a sterile container.

The method first comprises the step of washing the Plantago major and Mentha spicata plant material with purified water, such as distilled water, to remove any residue, such as pollen, dirt, insects, and other foreign debris remaining on the plant material, as shown by step 110. The washed plant material is then vertically stacked to allow the excess water to drip off, as shown by step 120. In one embodiment, the plant material is picked up by the stems and shaken vigorously to further remove excess water from the leaves. In another embodiment, the washed plant material is hung to allow the excess water to drip off. In this step, the plant material is not dried, instead the excess water is merely removed and the plant material is left damp before the next step in the method.

After the excess water has been removed from the plant material, the damp plant material is placed into a sealable freezer container and refrigerated in a freezer for approximately 48 hours at a temperature of approximately 15-20 degrees Fahrenheit, as shown by step 130. In one embodiment, a vacuum sealable freezer bag is utilized to store the plant material, such that all air can be removed from the interior volume of the bag while freezing the plant material therein. This freezing process causes the aqueous and non-aqueous liquids residing in the plant material to crystallize and expand, thereby rupturing the cellular membranes of the plant material. Thus, the trapped bioactive chemical agents contained in the cellular membranes of the plant material are released into the leaf structure of the plant material allowing them to be extracted therefrom.

Once frozen, the plant material is cut into smaller pieces with a pair of sterile scissors, as shown by step 140. This fragmented plant material is then heated in a sealed cooking utensil with 1 liter of purified water, such as distilled water or deionized water, at a temperature ranging between 130-140 degrees Fahrenheit for approximately 8-12 hours, as shown by step 150. In one embodiment, a slow cooker having a sealing gasket is utilized to heat the fragmented plant material. In another embodiment, the fragmented plant material is heated in 1 liter of double distilled water or demineralized water. This step of heating the fragmented plant material in the purified water macerates the plant material and extracts all water soluble chemicals from the plant material including solvents from the Mentha spicata plant material, thereby forming a solution comprising water-soluble chemicals and solvents. Moreover, heating the fragmented plant material in a sealed container prevents extracted chemicals having lower boiling points from evaporating and escaping the solution. The sealed container captures all gases, e.g., evaporated chemicals extracted from the plant material, therein, thereby enabling the gases to condense back into a liquid such that they remain a part of the solution. In total there are 12 solvents extracted from the Mentha spicata plant material during this step and they include acetone, benzyl alcohol, beta-phenethyl alcohol, beta-phenethyl-methylether, borneol, chloroform, dimethyl sulfoxide, ethanol, methanol, myrtenol, perillyl alcohol, and phenethyl alcohol. In one embodiment, the plant material is heated at 134.6 degrees Fahrenheit, for the purpose of preventing the boiling and evaporation of some of these solvents which contain lower boiling points and for extracting certain non-water soluble chemicals that become water soluble above 120 degrees Fahrenheit. During this heating period, the aforementioned extracted solvents in the resultant solution facilitate the extraction of non-water-soluble chemicals from both the Plantago major and Mentha spicata leaves.

After the solution is heated for approximately eight hours, the solution is allowed to cool by at least 35-40 degrees Fahrenheit to prevent the further evaporation of the extracted solvents that have low boiling points, as shown by step 160. Moreover, the solution is cooled to allow the evaporated chemical agents contained within the sealed cooking utensil to condense back into liquid form such that they remain in the solution. After cooling, all of the solid plant material remaining in the solution is pressed to extract the liquid remaining therein, as shown by step 170. This extracted liquid is then returned to the solution. In one embodiment, the resultant pressed matter weighs approximately 12.5 grams. The remaining solution is then filtered by passing it through a 26 micron mesh to remove all remaining particulate plant matter, as shown by step 180. Once filtered through the mesh, the resulting solution can be stored via freezing it into 10 mL ice cubes. In one embodiment, each ice cube comprises approximately 0.239 grams of Plantago major bioactive chemical agents and 0.019 grams of Mentha spicata bioactive chemical agents.

It is therefore submitted that the instant invention has been shown and described in various embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A composition for inhibiting or treating pathological conditions, the composition comprising an effective amount of a plurality of plant extracts from a Plantago major plant species and an effective amount of plurality of plant extracts from a Mentha spicata plant species.

2) A composition for inhibiting or treating pathological conditions, the composition comprising an effective amount of a plurality of plant extracts including acetonide, adenine, allantoin, apigenin, apigenin-7-glucoside, asperuloside, aucubin, baicalein, baicalin, benzoic acid, caffeic acid, catalpol, chlorogenic acid, cinnamic acid, D-glucose, D-xylose, ferulic acid, fumaric acid, geniposidic acid, gentisic acid, glucoraphanin, hispidulin, hydroxycinnamic acid, loliolide, luteolin, luteolin-7-O-beta-D-glucoside, neochlorogenic acid, nepetin, oleanolic acid, P-coumaric acid, P-hydroxybenzoic acid, phenolcarbonic acid, rhamnose, saccharose, salicylic acid, scutellarin, sitosterol, succinic acid, sulforaphane, syringic acid, syringin, tyrosol, ursolic acid, vanillic acid, acetic acid, acetoin, acetone, alpha-humulene, alpha-methyl-P-methylstyrene, alpha-pinene, alpha-terpineol, alpha-thujene, arginine, benzaldehyde, benzyl alcohol, benzyl isobutyrate, beta-bourbonene, beta-caryophyllene, beta-caryophyllene oxide, beta-elemene, beta-ionone, beta-limonene-1,2-oxide, beta-phenethyl alcohol, beta-phenethyl methyl ether, beta-pinene, beta-sitosterol, borneol, butyric acid, cadinol, camphene, carvacrol, L-carvone, carvone-10-acetate, carvone-8,9-oxide, chloroform, chlorophyll-A, chlorophyll-B, cineole, cis-carveol, cis-carvone oxide, cis-carvyl acetate, cis-jasmone, decan-1-ol, dehydrocarvacrol, delta-cadinene, diacetyl, dimethyl sulfide, dimethyl sulfoxide, diosmetin, diosmetin-7-glucoside, diosmin, ethanol, ethyl-N-valerate, eugenol, furfural, geraniol, heptan-3-one, hesperidin, hexan-1-ol, isoamyl isovalerate, isoleucine, jasmone, leucine, luteolin-7-O-beta-D-glucuronide, lysine, menthofuran, menthol, menthol acetate, methanol, methionine, myrtenal, myrtenol, neomenthol, niacin, oxalic acid, P-cymol, perillaldehyde, perillyl alcohol, phenethyl alcohol, piperitone, piperitone oxide, P-menth-trans-2,8-dien-1-ol, pomolic acid, propionaldehyde, riboflavin, rosmarinic acid, thiamin, threonine, thymol, tiglaldehyde, toluene, trans-carveol, trans-jasmone, terpinen-4-ol, valeric acid, valine, and vanillin.

3) The composition of claim 1, further comprising purified water.

4) A method of producing a composition for inhibiting or treating pathological conditions, comprising an effective amount of a plurality of plant extracts from a Plantago major plant species and an effective amount of plurality of plant extracts from a Mentha spicata plant species, the method comprising the steps of:
washing Plantago major plant material and Mentha spicata plant material with purified water;
removing excess water from the Plantago major plant material and the Mentha spicata plant material;
freezing the Plantago major plant material and the Mentha spicata plant material;
cutting the Plantago major plant material and the Mentha spicata plant material into fragments;
heating the Plantago major plant material and the Mentha spicata plant material in purified water in a sealed cooking utensil to form a resultant solution including Plantago major and Mentha spicata plant extracts;
cooling the resultant solution;
pressing the Plantago major plant material and the Mentha spicata plant material remaining in the resultant solution; and
filtering the fragmented plant material from the resultant solution.

5) The method of claim 4, wherein the Plantago major and Mentha spicata plant material is washed with distilled water.

6) The method of claim 4, wherein the excess water is removed by stacking the Plantago major and Mentha spicata plant material to allow the excess water to drip off.

7) The method of claim 4, wherein the excess water is removed by shaking the Plantago major and Mentha spicata plant material.

8) The method of claim 4, wherein the excess water is removed by hanging the Plantago major and Mentha spicata plant material.

9) The method of claim 4, wherein the Plantago major and Mentha spicata plant material is frozen for approximately 48 hours.

10) The method of claim 9, wherein the Plantago major and Mentha spicata plant material is frozen at a temperature ranging approximately between 15-20° F.

11) The method of claim 4, wherein the Plantago major and Mentha spicata plant material is cut using sterile scissors.

12) The method of claim 4, wherein the Plantago major and Mentha spicata plant material is heated in 1 liter of distilled water for approximately 8-12 hours.

13) The method of claim 12, wherein the Plantago major and Mentha spicata plant material is heated at a temperature ranging from approximately 130-140° F.

14) The method of claim 13, wherein the Plantago major and Mentha spicata plant material is heated at 134.6° F.

15) The method of claim 1, wherein the sealed heating utensil utilized to heat the Plantago major and Mentha spicata plant material is a slow cooker having a sealing gasket.

16) The method of claim 4, wherein the resultant solution is cooled by approximately 35-40° F.

17) The method of claim 4, wherein the Plantago major and Mentha spicata plant material is filtered from the resultant solution using a 26 micron mesh.

18) The method of claim 4, further comprising the step of freezing the resultant solution in 10 mL ice cubes for storage.
